(19) United States
(12) Patent Application Publication
(10) Pub. No.: US 2004/0241146 A1
(43) Pub. Date: Dec. 2, 2004

(54) Compositions, methods and apparatus for surgical procedures

(76) Inventor: Robert S. Biscup, Chagrin Falls, OH (US)

Correspondence Address: PEARNE & GORDON LLP, 1801 EAST 9TH STREET, SUITE 1200, CLEVELAND, OH, 44114-3108, US

(21) Appl. No.: 10/487,969

(22) PCT Filed: Aug. 27, 2002
(86) PCT No.: PCT/US02/27380

Related U.S. Application Data
(60) Provisional application No. 60/315,265, filed on Aug. 27, 2001.

(51) Int. Cl. A61K 45/00

(52) U.S. Cl. 424/93.7; 424/93.72

(57) ABSTRACT

A method, apparatus and kit for preparing and performing a surgical procedure includes providing a medicinal composition having an autologous tissue medium and a therapeutic agent. the medicinal composition is then delivered to a surgical site in a patient before closing to reduce postoperative pain, provide an interpositional membrane to prevent or inhibit the formation of scar tissue, and/or promote hemostasis. The medicinal composition is suitable for use in a variety of types of surgery, and has particular application in bone or tissue grating and orthopaedic surgery, expecially spine surgery.

### BACKGROUND OF THE INVENTION

1. Field of the Invention

The present invention relates to methods and apparatus for surgical procedures. More particularly, it relates to methods for surgical procedures including delivery of a medicinal composition that contains a therapeutic agent and an autologous tissue medium to a surgical site, and an apparatus for preparing an improved tissue or bone graft that incorporates said composition.

2. Description of the Related Art

Invasive surgical procedures are common practice in modern medicine, and are performed to ameliorate physiologic damage and defects of all kinds. Often, surgical procedures are performed to improve the quality of life of the patient, or even to save the patient's life. However, such procedures, even very successful ones, are not without their drawbacks.

Frequently, patients who undergo invasive surgical procedures experience significant postoperative pain which is particularly strong in the period immediately following surgery. Often, these patients are unable to get out of bed for days following surgery because of the severity of the pain. The degree of postoperative pain can depend on the surgical procedure performed, and also on the location of the surgical site.

In addition to postoperative pain, invasive surgical procedures sometimes result in other, more serious complications such as internal hemorrhage or postoperative infection at the surgical site. Such complications sometimes require one or more additional surgical procedures. Other complications do not manifest immediately during the postoperative period, but become more significant or even dangerous over time. For example, the potential for formation of internal scar tissue at the surgical site is a well known phenomenon associated with virtually any invasive surgical procedure. In many cases, this scar tissue does not cause significant medical concern or detrimental effects to the patient. However, in some cases, particularly in neurosurgical and orthopaedic procedures, the formation of this scar tissue can be devastating.

Spine surgery is one type of surgery where the above-noted complications can be particularly severe. Spine surgeons perform surgical procedures to repair or correct defects in the spinal column. Always of concern to the spine surgeon both during and after any spine surgery, is the preservation of the integrity of the spinal nerves and the dura that encloses those nerves (which together make up the spinal cord) at the center of the spinal column, and of the nerves that connect thereto and/or extend from the spinal cord through the vertebrae to control other body systems. During any spine surgery, the spine surgeon must be extra careful to avoid injuring such nerves, often moving one or more as he works on the bones and discs that make up the spinal column. Once a procedure is complete, the surgeon carefully restores and cleans the surgical field prior to closing.

Unfortunately, sometimes the above-noted complications can spoil the good work of even the most careful and skillful spine surgeon. In the immediately postoperative period, the swelling or formation of a hematoma can exert significant pressure on the nerves or other tissues at the surgical site. This increased pressure can cause severe, even debilitating pain, partial or total paralysis, or even death if not immediately repaired. To repair these complications, a second surgical procedure may be required, which itself can result in the same or similar complications once again.

In still other patients, some of whom may weather the immediately postoperative period without serious complications, the formation of scar tissue at the surgical site can be a slow process that does not result in any noticeable effects for months or even years following the surgery. However, once it manifests, such scar tissue can exert significant pressure on the spinal cord or its associated nerves. This pressure can produce many or all of the terrible effects mentioned in the preceding paragraph. In fact, the formation of scar tissue following spinal surgery can evolve into chronic problems for the patient which cannot be permanently repaired by revision surgery. This is because in patients who are predisposed to form significant scar tissue, revision surgeries to remove the scar tissue will provide only a temporary solution until the future formation of new scar tissue at the surgical site. The result can be a cycle of repeated invasive surgeries to temporarily relieve the patient of excruciating or debilitating pain or disability, only to have to return again to the operating room for the same procedure some months or years down the road.

Each trip to the operating room involves a separate, additional invasive procedure performed under general anesthesia, increasing the chances for life-threatening or debilitating complications to occur or recur.

Though the foregoing has been described with respect to spine surgery, the mentioned complications are not unique to spine surgery and can and do occur following many other types of surgical procedures, including other orthopaedic and non-orthopaedic procedures. In particular, patients that have received tissue grafts (including bone grafts as well as other tissue grafts, such as soft tissue grafts) can be particularly susceptible to postoperative infection as well as other of the above-described complications. These patients could benefit significantly from a composition that is effective to inhibit or suppress these complications being applied directly to the surgical or graft site.

To help minimize pain during the immediately postoperative period, it has been proposed to introduce a composition containing a pain medication at a surgical site. For example, duramorphine has been dispersed in Avitine powder (heterologous collagen delivery vehicle) to form a pain paste to be applied to a spinal surgical site following spine surgery to alleviate pain. This pain paste suffers from the drawback that the carrier medium is a non-autologous (heterologous) material to the patient's body which can result in immunosuppressant rejection and other complications.

Accordingly, there is a need in the art for a method to minimize or eliminate the occurrence of some or all of the aforementioned postoperative complications. Such a method preferably can be easily performed prior to closing or after closing, and will assist or speed healing at a surgical site. Most preferably, such a method will not itself introduce any postoperative complications to the patient, for example, immunosuppressant rejection of introduced compositions.

### SUMMARY OF THE INVENTION

A method of performing a surgical procedure on a patient is provided. The method includes the steps of providing a medicinal composition that comprises an autologous tissue medium, and delivering the medicinal composition to a surgical site in the patient. The medicinal composition has at least 50 weight percent autologous tissue medium.

Another method of performing a surgical procedure on a patient is provided that includes the steps of preparing a tissue graft material, implanting the tissue graft material to a graft site in the patient to provide a tissue graft, and applying a separate layer of a medicinal composition over the tissue graft. The medicinal composition includes an autologous tissue medium.

An apparatus for preparing tissue or bone graft material is also provided. The apparatus includes a tissue or bone comminuting unit that has a scalloped drum. The drum has a first drum end, a second drum end, a drum wall having an interior wall surface and an exterior wall surface, and a number of scallops extending from the exterior wall surface of the drum wall. Each of the scallops has an associated aperture through the drum wall.

A kit for preparing a tissue or bone graft is also provided. The kit includes a tissue or bone comminuting unit and a mixing unit. The comminuting unit has a scalloped drum having a first drum end, a second drum end, a drum wall having an interior wall surface and an exterior wall surface, and a number of scallops extending from the exterior wall surface of the drum wall. Each of the scallops has an associated aperture through the drum wall. The mixing unit has an auger that is mounted or mountable within a mixing chamber of the mixing unit. The mixing unit is adapted to receive within the mixing chamber comminuted bone or tissue fragments from the comminuting unit.

A medicinal composition for application to a surgical site in a patient is also provided. The composition includes autologous tissue medium and at least one therapeutic agent which could be, for example, a pain medication, an anti-scarring medication, and/or a steroid.

### BRIEF DESCRIPTION OF THE DRAWINGS

FIG. 1 is a side perspective view of a tissue or bone comminuting unit according to the invention.

FIG. 2 is a side view, partially in section, of the comminuting unit of FIG. 1.

FIG. 3 is an end view in cross section, of the comminuting unit of FIG. 1.

FIG. 4 is a side view, partially in section, of a mixing unit having a plurality of injector ports according to the invention.

FIG. 5 is a schematic view showing the mechanism of detachment of a tissue graft material delivery tube from the mixing unit of FIG. 4 and subsequent attachment or mounting to an injection gun for delivery of an improved bone tissue according to the invention.

FIG. 6 is a schematic side view, partially in section, of the injection gun shown in FIG. 5, with the graft material delivery tube mated therewith.

FIGS. 7-9 are side views of different graft material delivery tubes according to the invention.

### DETAILED DESCRIPTION OF THE PREFERRED EMBODIMENTS OF THE INVENTION

As used herein, when a range is given, such as 5-25 (or 5 to 25), this means at least 5 and, separately and independently, not more than 25.

The invention provides a method of performing a surgical procedure that includes delivery of a therapeutic agent to a surgical site in the form of a medicinal composition. Preferably, the medicinal composition is in a gel state, and includes at least the following two components: an autologous tissue medium and at least one therapeutic agent. Preferably, the autologous tissue medium is 50-99, preferably 60-99, preferably 70-99, preferably at least 80, weight percent of the medicinal composition, balance therapeutic agent(s) and/or other components as will be described below. Alternatively, in certain preferred embodiments as will become evident below, the medicinal composition can be devoid of any therapeutic agent. As used herein, the term ‘therapeutic agent’ means any agent, compound, ion, salt, chemical species, molecular species, medicament, or other component of a medicinal composition that is not a living organism or cell, and which is provided to the medicinal composition to achieve a specific therapeutic result in a patient. Therapeutic agents include, but are not limited to, medications, enzymes, chemicals, cell nutrients and growth factors.

Also as used herein, the term ‘gel’ means a material that is flowable at 22° C. and 1 atm, but sufficiently viscous and having sufficient adhesive and cohesive properties as not to substantially flow spontaneously (e.g. gravity-induced flow) without the application of an external pressure to overcome the material's flow-resistance due to the above-stated properties. Preferably, as used herein, a gel has a viscosity of at least 10 cP, preferably at least 100 cP, preferably at least 1,000 cP, preferably at least 10,000 cP.

The invention includes: preparation and application, in a suitable manner, of a medicinal composition onto (a) a surgery-specific situs at the completion of or immediately after surgery, (b) a specific internal situs prior to surgery, or (c) an internal situs or body locus as part of a non-surgical (or minimally invasive surgical [MIS]) course of treatment. The medicinal composition (which can be an engineered mixture of an autologous fluid or soft tissue carrier medium and specific therapeutic agents) is applied to the surgical site to help the patient avoid pain, minimize bleeding or infection, and promote homeostasis. Additional targeted benefits include prevention of scar formation around neurological strictures. These and other benefits of providing the medicinal composition to a surgical site are described in greater detail below. The invention has applications in most types of surgery, but with specialized formulations and/or with specifically engineered materials for a targeted application, has particular application for spine surgery, orthopaedic surgery, neurosurgery, pain management, plastic surgery and ENT surgery.

Autologous tissue medium is a material that is prepared from autologous material extracted from the patient's body, preferably from the patient's blood, serum and/or blood-forming tissue, such as bone marrow. Less preferably the autologous material can be derived or extracted from the patient's fat or adipose tissue, less preferably from umbilical blood or liquefied nerve tissue, less preferably other autologous tissue. The autologous tissue medium preferably contains only material that is autologous to the patient's body in which it will be used. Preferably, the autologous material is extracted intraoperatively from a patient's body during a surgical procedure.

In the invented medicinal composition, the autologous tissue medium is the substrate or carrier in which therapeutic agents and/or other components are added, dissolved, dispersed, etc. In other words, the autologous tissue medium acts as the delivery vehicle for contained therapeutic agents and other medicinal composition components. Alternatively, in certain preferred embodiments the autologous tissue medium can be used alone (devoid of therapeutic agents and/or other components) to provide certain benefits to a patient when applied to a surgical site as will be further described.

The autologous tissue medium is preferably autologous platelet gel, preferably autologous platelet gel having autologous growth factors (AGFs) added thereto and concentrated therein, which is referred to herein as AGF-platelet gel. Preferably, the platelet gel has a platelet concentration in the range of 1×10⁹ to 1×10¹², preferably at least 4×10¹⁰, preferably at least 5×10¹⁰, preferably at least 6×10¹⁰, preferably at least 7×10¹⁰, preferably at least 8×10¹⁰, preferably at least 9×10¹⁰, preferably at least 1×10¹¹, preferably at least 1.1×10¹¹, preferably at least 1.2×10¹¹, preferably at least 1.3×10¹¹, platelets per mL of the gel. When AGF-platelet gel is used, the AGFs added thereto preferably include, but are not limited to, TGF-β and PDGF, and are selected to provide the most efficacious platelet gel for the desired application at a surgical site; e.g. pain mitigation, hemostasis, scar tissue suppression, etc., or any combination of these.

The autologous platelet gel is preferably prepared as a gel concentrate, preferably via known or conventional techniques utilizing known machines, such as a platelet gel concentrator machine from Cross/Interpore or the Symphony system from DePuy/Johnson & Johnson. There are a variety of conventional methods of securing autologous gel concentrate which include collecting an autologous blood product or fat product obtained intraoperatively from the patient in various quantities depending on the application or need. This type of product can be obtained or derived from direct blood aspiration, from the known Cell Saver blood salvage machine during a surgical procedure, or from a liposuction device as known in the art. With respect to blood, whole blood can be utilized directly from a Cell Saver machine during a surgical procedure, or it can be fractionated through the Cross/Interpore or DePuy devices mentioned above. Less preferably, whole blood can be fractionated via any other known or conventional concentrating device that is effective to produce a concentrated autologous platelet gel or other tissue gel whose concentration is substantially constant. Concentrated platelet gels contain additional blood serum components such as various proteins and growth factors. Less preferably, autologous tissue media made from other tissues (e.g. bone marrow, fat, adipose tissue, etc.) can be prepared via known or conventional techniques.

The autologous tissue medium of the present invention is preferably made from primary autologous material, which means materials derived directly from the patient's own extracted or harvested tissue (i.e. blood, serum, marrow, fat, or other of the patient's tissues such as soft tissues), preferably intraoperatively. Less preferably, the autologous tissue medium of the present invention is made from secondary autologous materials, which means materials that are separately generated or replicated from the patient's extracted materials as described above via known or conventional techniques, e.g. in vitro tissue growth or culture, cloning, replication, leeching, or otherwise. Primary autologous materials are preferred in the invention because they can be extracted from the patient quickly and intraoperatively, and do not require significant time to complete. The preparation of autologous tissue medium from primary autologous materials can be done intraoperatively in under 90 minutes, preferably under 60 minutes, preferably under 50 minutes, preferably under 40 minutes, preferably under 30 minutes, preferably under 20 minutes, preferably under 15 minutes, preferably under 10 minutes, preferably under 5 minutes, following the extraction of the primary autologous materials from the patient. However, for a surgery that is scheduled in advance, it may be preferable to prepare the autologous tissue medium from secondary autologous materials generated using primary autologous materials donated by the patient in advance of the surgery. This may allow the preparation of a significantly larger quantity of autologous tissue medium than could otherwise be produced intraoperatively directly from the patient's extracted primary autologous materials. For example, when AGF-platelet gel is used as the autologous tissue medium, 25 cc of blood will yield only about 5 cc of AGF-platelet gel, (and 200 cc of blood typically will yield only about 50 cc of AGF-platelet gel). Thus, the short supply of AGF-tissue gel can be overcome if additional secondary autologous materials are generated prior to surgery for the preparation of additional AGF-platelet gel.

Once the autologous tissue medium has been prepared, it is combined or mixed with the therapeutic agent or agents in volume or weight ratios known in the art (or which can be ascertained by a person of ordinary skill in the art without undue experimentation) to form the desired medicinal composition. The therapeutic agent or agents is/are selected to facilitate a desired medicinal or therapeutic effect at the surgical site as is more fully described below. In a preferred embodiment, the therapeutic agent is first provided in micro-capsules or spansules which are conventional in the art for the time-release administration of a medication. In this embodiment, the spansules containing the therapeutic agent are mixed with the autologous tissue medium as described above, and preferably mechanically agitated very gently to avoid rupturing the spansules and premature delivery of the therapeutic agent within. Time-release spansules are particularly preferred to administer a pain medication (such as morphine) internally at the surgical site. This will provide the patient long-lasting, consistent pain relief for the duration of the immediately postoperative period instead of a single high initial dose that wears off prematurely.

The medicinal composition (containing the therapeutic agent(s) dispersed or mixed in the autologous tissue medium) is preferably applied internally to the surgical field following a surgical procedure and immediately prior to closing. The medicinal composition can be provided with a variety of therapeutic agents, as well as other components, to achieve a desired therapeutic effect. The therapeutic agents initially may be liquid, gaseous or solid in their pre-mixture state prior to being combined with the autologous tissue medium to form the medicinal composition of the present invention. The medicinal composition itself can be a true solution, a suspension, a slurry or other suitable physical form so long as at least 10, preferably at least 20, weight percent of the finished medicinal composition is in a non-solid, e.g. liquid (or gel), flowable state in order to facilitate transmission of the medicinal composition through a hollowed transmission device, such as a pipe, tube, syringe, hose or other similar contrivance. Several examples of the use of a therapeutic agent according to the invention are provided below. These examples represent preferred embodiments of the invention, but are not an exclusive list.

Pain Gel—The autologous tissue medium (such as bone marrow concentrate or gel or AGF-platelet gel) is mixed with preservative free and concentrated pain medication (e.g. epidural morphine or Fentanyl) and/or a steroid preparation such as dexamethasone or solumedrol to provide a pain gel. Optionally and preferably, the pain gel can also include corticotropic preparations and/or a nerve impulse stabilizing agent such as neurontin. Other conventional components can be added as well. Immediately following a surgical procedure, the pain gel is applied over the surgical field prior to closing. As discussed above, the pain medication is preferably provided in time-release spansules as part of this application. Studies have shown the significant effectiveness of other epidural pain “paste” materials to alleviate post-surgical pain, accelerate patient ambulation, decrease hospital stay, minimize use of narcotic medication, and return faster to exercise and work. The invented pain gel is believed to have similar properties. Furthermore, the invented pain gel utilizes a carrier medium that is autologous to the patient's own body, and hence presents zero risk of immunosuppressant rejection or related complications. In the case of spinal surgery, the invented pain gel is particularly useful. Following the surgical procedure, the invented pain gel is provided, preferably copiously, within the epidural space adjacent the surgical site, preferably in contact with, covering or substantially covering the dura. In this manner, the pain gel can deliver the therapeutic agent (pain medication) directly to the spinal nerves, and significantly reduce postoperative pain. The medicinal composition preferably has the following composition as shown in table 1 to provide the pain gel as described in this paragraph. All values in table 1 are weight percents.

TABLE 1
Preferred composition of medicinal composition for pain gel according
to the invention

| Autologous platelet gel | 50-99, preferably |
|---|---|
| | 60-90, preferably |
| | 70-80 |
| Pain medication (preferably | 1-20, preferably |
| duramorphine or Fentanyl) | 2-15, preferably |
| | 5-10 |
| Steroid (preferably | 0-15, preferably |
| dexamethasone or solumedrol) | 2-15, preferably |
| | 5-10 |

Hemostatic Agent—Primary or revision spinal surgery can be associated with significant epidural bleeding and large volumes of blood loss. Often, patients will continue to drain from their surgical wounds for several days postoperatively. The invention can significantly lower post-surgical bleeding and drainage thereby decreasing morbidity and the need for additional blood transfusions, resulting in shorter hospital stays. When hemostasis is the only concern at a surgical site, the autologous tissue medium can be used alone, without the addition of a therapeutic agent. Specifically, again in the case of spine surgery, autologous tissue medium that is prepared as described above can be provided into the epidural space immediately following the surgical procedure to promote hemostasis; i.e. minimize or prevent excessive epidural bleeding postoperatively. It should be noted that adding pain medication and other components as described in the preceding paragraph will not diminish the hemostatic capability of the resulting composition. Rather, the pain gel as described will serve a dual function; to prevent/minimize significant postoperative pain as well as to prevent excessive bleeding or hemorrhage at the surgical site. For the foregoing reason, it will usually be preferred to include at least a pain medication as a therapeutic agent. This is especially true for spine and other orthopaedic surgeries, as well as other surgeries at or near a nerve plexus where pain can be particularly severe. However, in other surgical procedures, where postoperative pain is not so great a concern, the autologous tissue medium can be used alone as a hemostatic agent.

Interpositional Membrane—Post-surgical scarring is of particular concern during spinal surgery. It is believed that providing a quantity of the autologous tissue medium (with or without additional therapeutic agents) in the epidural space to coat the dura following spine surgery will prevent or substantially inhibit the formation of scar tissue, or the adherence of scar tissue to the dura. The autologous tissue medium provides a layer of material between the dura and paraspinal tissue thereby decreasing attachment of paraspinal muscle to dura and nerve roots, as well as other fibrous scar tissues, that could cause tethering and restriction of motion. In a preferred embodiment, when used for this application, the autologous tissue medium is combined with a steroid as therapeutic agent, such as cortisone, dexamethasone, or solumedrol, or a liquid anti-inflammatory agent such as a cox-2 inhibitor or similar agent, since it has been shown that these and similar agents can be used to slow or inhibit the formation of scar tissue postoperatively. It is noted that a medicinal composition comprising the pain medication described above (whether time release or not) and the steroid or other preferred component as described in this embodiment preferably will provide all three of the beneficial functions described in this and the preceding two paragraphs; i.e. pain mitigation, hemostasis and scar tissue suppression at the surgical site. Such a medicinal composition is of particular use following spine surgery, where the effects of postoperative pain, hemorrhage or hematoma, and scar tissue formation all can be devastating and debilitating, whether acute or chronic.

Other therapeutic agents which are known in the art can be combined with autologous tissue medium to form medicinal compositions according to the invention having unique properties. By way of example, the therapeutic agent can be any pharmacologically or homeopathically appropriate compound that is specifically chosen to achieve an intended purpose, such as, e.g., analgesics, anti-inflammatory agents or compounds, healing stimulants and/or inhibitors, osteogenic stimulants and retarders, etc., all of which are known or conventional in the art. The entire range of therapeutic agents that can accelerate or retard or lessen or heighten physiologic responses can be used and combined as appropriate with an autologous tissue medium as described above to provide a medicinal composition according to the present invention.

Optionally, a cellular component (e.g. autologous living cells) can also be added to the medicinal composition. When present, the cellular component or components is/are selected to achieve or promote a desired physiologic action or response in the patient. For example, when the medicinal composition is used to augment bone grafting (described below), the medicinal composition can be provided with cells that promote or participate in osteogenesis, such as osteoblasts, osteoclasts, other cells responsible or necessary for angiogenesis, etc. If infection is of concern at a graft or surgical site, it may be desirable to provide white blood cells in the medicinal composition. Most preferably, when a cellular component is present, such component is autologous to the patient that will receive the medicinal composition. The cells are harvested from the patient via known techniques, preferably intraoperatively, and then combined in the medicinal composition. Alternatively, the cells can be harvested from the patient in advance of surgery (e.g. for a scheduled or elective surgery) and replicated or grown in vitro (e.g. cultured) to produce an expanded population of the cells. This expanded population is then combined with the medicinal composition and applied to a surgical site during surgery and will provide an enhanced physiologic effect corresponding to the expanded population of cells in the composition (e.g. accelerated bone generation, healing, angiogenesis, etc.).

The invention is preferably used in spine surgery. Alternatively the invention can be used in other surgical disciplines such as ENT and plastic surgery. In particular, the invention can be used in the following applications: bone grafting, soft tissue grafting, bone healing, soft tissue healing, muscle regeneration, in vitro cell culture growth stimulation using harvested autologous cells and fractionated growth factors, and intradiscal injection and regeneration (or strengthening) of weakened, damaged, or degenerating intervertebral disc structures (annulus fibrosis & nucleus pulposis). The invented composition can also be used to enhance bone fusion and in other bone applications, e.g. via incorporation of autologous tissue medium-coated bone or bone substitute material into fusion cages and/or bioactive materials. The autologous tissue medium as described above (whether or not combined with a therapeutic agent to provide a medicinal composition) can be introduced into a patient to ameliorate bone defects or to accelerate bone healing via percutaneous injection, i.e. into delayed bone healing conditions (prior fusions, fractures, radiation treatment for tumors, etc.), and intraosseous (transpedicular) injection for bone augmentation in osteoporosis. Other applications which are not described herein but which are within the scope of the present invention will be apparent to the person of ordinary skill in the art.

A preferred method of using and applying the medicinal composition according to the invention is described below. Though the invention is preferably performed during, and is described herein largely with respect to spine surgery, it will be understood by persons of ordinary skill in the art that the following method can be practiced with equivalent or analogous effect in other types of surgical procedures, for example those described in the preceding paragraph.

First, the autologous tissue medium is prepared from autologous material that is extracted from the patient, preferably intraoperatively. The autologous material is preferably primary autologous material as described above. Less preferably, (though perhaps more preferably in the case of elective or other surgery that is scheduled in advance), the autologous material can be secondary autologous material that is generated or cultured using the patient's primary autologous material. The remainder of the method will be described for primary autologous material that is extracted intraoperatively.

The primary autologous material can be collected intraoperatively via known means and techniques, e.g., using an aspiration device such as a syringe and needle, the well known Cell Saver blood salvage machine, a liposuction device, etc.

In a preferred embodiment, when the autologous tissue medium is to be AGF-platelet gel, the autologous material is extracted via needle aspiration from the patient's blood system, or otherwise from the Cell Saver blood salvage machine described above. Next, the extracted whole blood is treated via known techniques using known or conventional machines or devices as described above to concentrate the platelets therein to provide a concentrated AGF-platelet gel. The remaining materials, including residual macrophages, erythrocytes, other blood components and cells, and liquid plasma, can be discarded or, more preferably, they can be reinfused into the patient. The AGF-platelet gel is combined or mixed via conventional techniques with one or more therapeutic agents to provide a medicinal composition having the desired therapeutic properties. (Optionally, e.g. when hemostasis is of sole or primary concern, the AGF-platelet gel can be used without adding any therapeutic agents). The medicinal composition is then applied over the surgical site, as described above, preferably after completion of the surgical procedure but prior to closing.

The invention includes delivery of the medicinal composition directly onto the surgical site, as well as delivery from outside the specific surgical site (or even from outside the body through a percutaneous incision), and injection/delivery of the composition directly to the situs through a rigid or flexible tube or cylinder, including a hollowed injection needle. One can directly apply the desired composition to the situs during open surgery, either prior to surgery or at the commencement of the surgical procedure (to achieve a desired result) or, more probably, at the completion of the surgical procedure to help minimize post-surgical pain/trauma/recovery-delay prior to closing. Further, the invention can be used as an alternative to surgery or to treat other symptoms where surgery is not intended (e.g. pain management applications).

In a further preferred embodiment, the invention is applied to provide an improved tissue graft that contains a therapeutic agent as described above. As used herein, a tissue graft can be a graft for any suitable body tissue or material for which grafting is or can be performed. For example, a tissue graft can be a soft tissue graft, vascular graft, bone graft, bone marrow graft, or any other graft that is known or conventional to persons of ordinary skill in the art. This method is particularly useful when a patient is undergoing spinal or orthopaedic surgery and will need a bone graft. In this embodiment, a bone graft is provided that has added to it a quantity of the invented medicinal composition to achieve a particular therapeutic purpose. For example, the therapeutic agent can be or include various osteoblastic nutrients (such as glucose, amino acids and/or dissolved oxygen) or growth factors to promote osteogenesis and bone fusion at the graft site. In addition, pain medication, including a time-released pain medication, can be provided as a therapeutic agent. Other therapeutic agents of known utility can be incorporated as well.

The method of preparing and implanting a tissue graft material (such as bone graft material) preferably proceeds as follows. First, the graft site is prepared to receive the graft material in a conventional manner. The graft material itself is prepared, also in a conventional manner using known techniques. The graft material is preferably autologous graft material that is taken or extracted from the body of the patient that will receive the graft. For a bone graft, bone graft material is preferably autograft material harvested from the patient (e.g. from the iliac crest, vertebra, long bones of limbs, etc.). Next, a volume of the patient's blood or aspirated bone marrow (preferably primary autologous material) is used to provide a certain amount of autologous tissue medium, preferably AGF-platelet gel, less preferably other tissue gel such as marrow-derived tissue gel as described above. Next, the desired therapeutic agent(s) is/are combined and mixed with the autologous tissue medium to provide the desired medicinal composition. The graft material is then mixed with the medicinal composition to provide an improved graft material. (For bone grafting, improved bone graft material can be prepared, e.g., by combining the medicinal composition with chips or pieces of bone and other conventional materials (e.g., thrombin) and mixing to form a bone paste). Subsequently, the improved graft material is implanted in the patient at the graft site to form a tissue graft. In the case of spine surgery, the tissue graft can be a bone graft, for example to provide a spinal fusion, with or without cages. Optionally and preferably, a separate layer of the medicinal composition is applied over the newly implanted graft at the graft site, for example, placed on top of the nerves and/or dura at the site (particularly in spine surgery). This separate layer of medicinal composition will help alleviate pain during the immediately postoperative period, as well as act as an interpositional and hemostatic membrane (described above) to prevent or minimize scar formation to the implanted graft as well as postoperative hemorrhage. It should be noted that the medicinal composition that is added to (mixed with) the graft material does not need to be the same medicinal composition that is used to provide the separate layer over the implanted graft. For example, both medicinal compositions can be made from the same sample of autologous tissue medium, but using different therapeutic agents (e.g. osteoblastic nutrients for improved bone graft material, and pain medication for the separate layer). Alternatively, the separate layer need not contain a therapeutic agent at all.

The inventor herein has observed that patients who have undergone spinal surgeries to repair bony defects in the spinal column, such as spinal fusion, have experienced significantly less postoperative pain, ambulated sooner, and tended to heal at an accelerated rate when the invented medicinal composition was applied within the epidural space immediately following the surgical procedure. The use of autologous platelet gel as the autologous tissue medium in the medicinal composition has proven quite effective at enhancing the rate of healing of patients who have undergone spinal fusion; thus autologous platelet gel has been found to biologically enhance the patient's healing response following a spinal fusion surgery.

When the medicinal composition is to be used in tissue grafting applications, e.g., such as bone grafting to provide a spinal fusion as described in the preceding paragraph, the improved tissue graft material comprising the invented medicinal composition is preferably prepared using the following apparatus and methodology. Referring now to the figures, the preferred apparatus includes a bone or tissue comminuting unit 14, a mixing unit 30, at least one graft material delivery tube 48 and an injection gun 50.

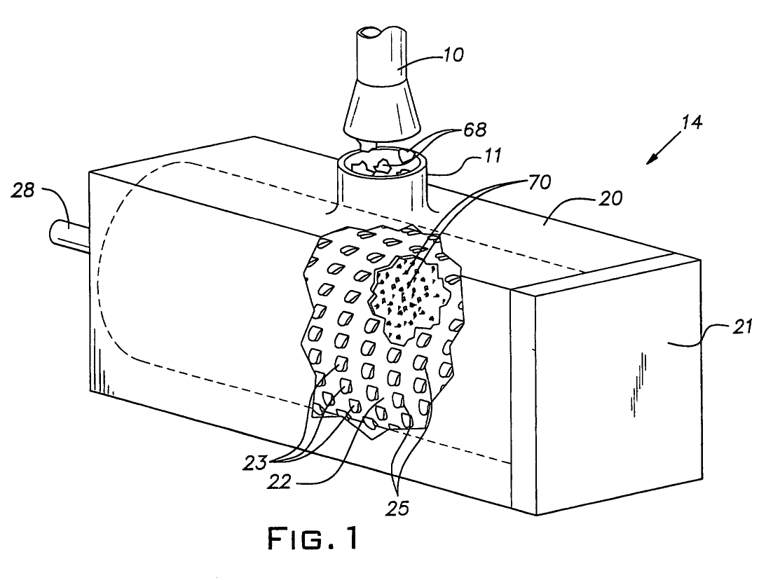

FIGS. 1 to 3 show a tissue processing, cutting or comminuting unit 14 according to the invention. The comminuting unit 14 has a casing 20 that encloses a scalloped metal drum 22 having a drum wall (preferably aluminum) with an interior wall surface and an exterior wall surface, and teeth or scallops 23 extending from the exterior wall surface of the drum wall. The casing 20 and scalloped drum 22 define a cutting chamber 18 therebetween, and the scalloped drum 22 defines a bone or tissue fragment collection chamber 26 therein. Each scallop 23 has an associated hole or aperture 25 through the drum wall. The scalloped drum 22 preferably has a first, substantially closed drum end 27 and a second, open drum end 29. The casing 20 also preferably has first and second substantially closed and open ends 84 and 85 respectively adjacent the like ends of the scalloped drum 22. The scalloped drum 22 is supported and adapted to be rotated within the casing 20, and is preferably fitted with a drill connector 28 at the first end thereof. The drill connector 28 can extend through the wall of the casing 20 or it may be accessible from the outside through a hole in the casing wall. In either case, the drill connector 28 is adapted to mate with a drill (not shown), which may or may not be fitted with a suitable drill bit to mate the connector 28.

The casing 20 preferably has a port 11 formed in the wall thereof adjacent the exterior wall surface of the drum 22. As best seen in FIG. 3, most preferably the comminuting unit 14 includes shield members 15 cantilevered from the interior surface of the casing 20 adjacent the port 11. The shield members 15 preferably extend substantially axially toward the drum 22, but stop short of coming into contact with the scallops 23 during rotation of the drum. The shield members 15 substantially prevent bone or other tissue material 68 from falling around the drum 22 to the bottom of the casing 20 during a comminution operation as described below. In this manner, all or the vast majority of the tissue material 68 introduced into the comminuting unit 14 through the port 11 is comminuted against the drum 22 and scallops 23 using the plunger 10 to produce comminuted tissue fragments within the collection chamber 26 of the drum. In practice, when the tissue graft is to be a bone graft, the tissue material 68 can include bone pieces, chips, chunks or nuggets that are introduced into the cutting chamber 18 through the port 11 and pressed against the surface of the drum 22 and the scallops 23 via the plunger 10 to provide comminuted bone fragments 70 as further described below. Alternatively, when the tissue graft is to be a soft tissue or other tissue graft, the tissue material 68 includes the appropriate graft precursor materials for the intended graft as known in the art. These precursor materials are then comminuted via the scalloped drum 22 and scallops 23 as described above.

For simplicity, the remainder of the method of using the invented apparatus will be described with respect to preparing a bone graft material for bone grafting. However, it will be understood by a person of ordinary skill in the art that when another tissue graft material is to be prepared, one would proceed similarly as described below except substituting the necessary precursor material for bone chunks to produce the desired tissue graft material. Such tissue graft material would similarly be applied to a graft site as also described below.

The scalloped drum 22 is provided with a plunge plate 24 within the collection chamber 26. Preferably, the plunge plate 24 slidably engages the interior wall surface of the drum 22. The plunge plate 24 is adapted to slide through the collection chamber 26 from the closed drum end 27 toward the open drum end 29 in order to force comminuted bone fragments 70 out of the drum 22 and into a mixing unit 30 described below. The closed drum end 27 of the drum 22 is preferably provided with an opening 16 therethrough to allow a plunge rod (not shown) or other suitable contrivance to be inserted therethrough, and to push against the plunge plate 24 in order to force comminuted bone fragments 70 out the open drum end 29. Optionally, the drill connector 28 has a hole therethrough to accommodate the plunge rod so that the plunge rod can be inserted from outside of the casing 20 adjacent the closed drum end 27 of the drum 22, to engage the plunge plate 24 in the chamber 26. Less preferably, the plunge plate 24 can be advanced toward the open drum end 29 of drum 22 via any other suitable mechanism.

Preferably, the casing 20 of comminuting unit 14 is provided with a removable cover plate 21 to seal off the open drum end 29 during a comminuting operation. Once a comminuting operation is complete, the cover plate 21 is removed, and the comminuting unit 14 is mated to a first end of the mixing unit 30 which will now be described.

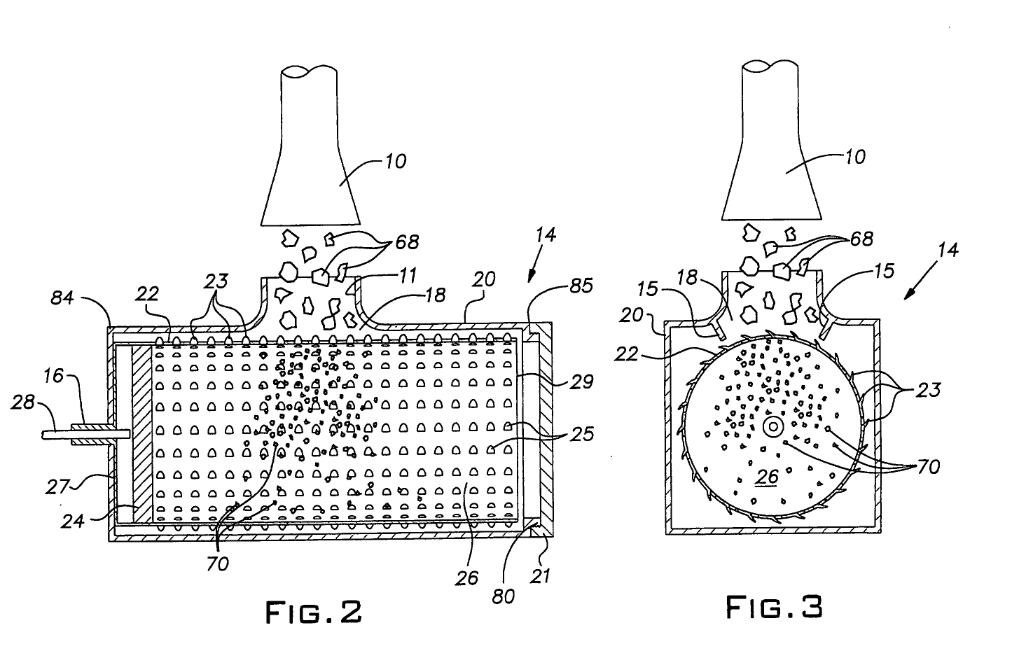

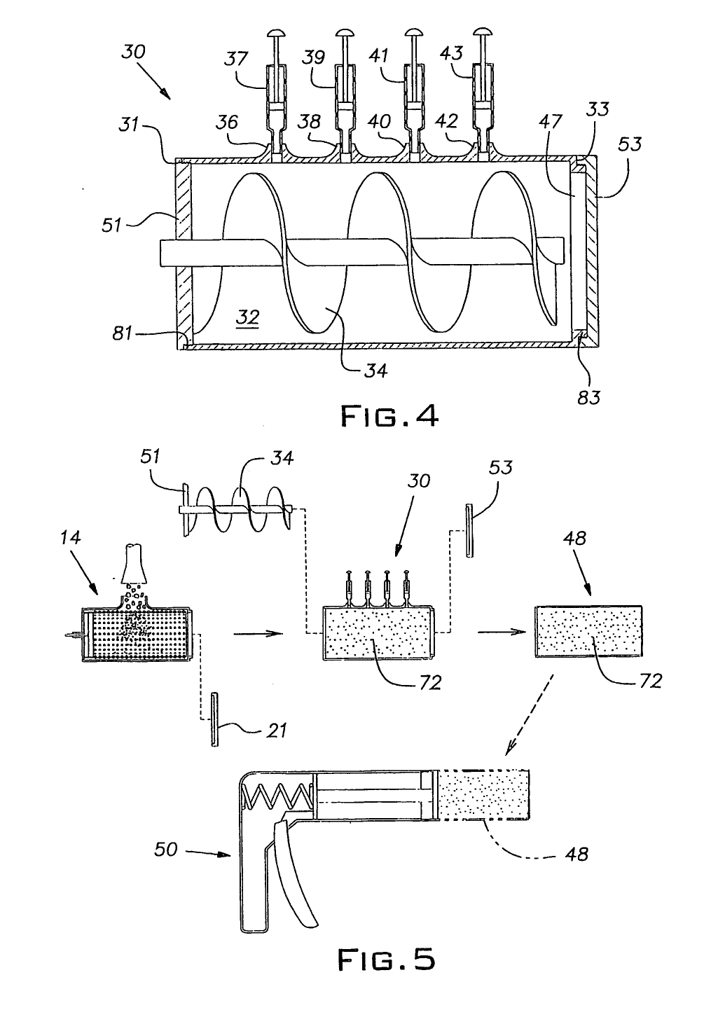

The mixing unit 30 preferably includes an enclosure which defines a mixing chamber 32 therein, and has a first mixing end 31 and a second mixing end 33. Less preferably, the mixing unit need not be enclosed; e.g. the mixing unit can have an open top. The first mixing end 31 is adapted to mate with the second open end 85 of the comminuting unit 14 adjacent open drum end 29, and the second mixing end 33 has an exit port 47 that is adapted to mate with a graft material delivery tube 48 as hereinafter described. As best seen in FIGS. 2 and 4, the comminuting and mixing units 14 and 30 are preferably mated by engaging an inwardly extending lip or flange 80 at the open end 85 of the comminuting unit 14 and an overhang portion 81 at the first mixing end 31 of the mixing unit 30. Also preferably, the delivery tube 48 has an inner diameter properly sized to accommodate the port flange 83 of the exit port 47 of the mixing unit 30, and to securely retain the delivery tube 48 on the flange 83. Less preferably, the comminuting and mixing units 14 and 30, and the mixing unit and delivery tube 30 and 48 can be mated via any other suitable means.

The mixing unit 30 preferably includes a first cover 51 and a second cover 53 for sealing the first mixing end 31 and exit port 47 respectively of the mixing unit during a mixing operation. The mixing unit 30 has a screw or auger 34 rotatably mounted or mountable in the mixing chamber 32. The auger 34 is preferably made from metal, preferably aluminum or surgical steel. Auger 34 is preferably driven in rotation by a drill that is fitted with a suitable drill bit and connector, via a hand crank, or other suitable means (not shown). Optionally and preferably, the auger 34 can be mounted to the first cover 51 so that it is introduced into the mixing chamber 32 upon sealing the mixing unit 30 after bone fragments 70 are provided therein from the cornminuting unit 14. The mixing unit 30 has a number of injector ports 36, 38, 40, 42 therein, which are preferably adapted to mate with syringes 37, 39, 41 and 43 respectively. Alternatively, ports 36, 38, 40 and 42 are each provided as a cylindrical housing that is molded or permanently attached to the body of the mixing unit 30, and which is adapted to receive a plunger therein to force the contents thereof into the mixing chamber 32. Though four injection ports are shown, the mixing unit 30 can have any number of injection ports to accommodate different numbers of graft material components as described further below, e.g. 1-5, 1-10, or 1-15 injection ports.

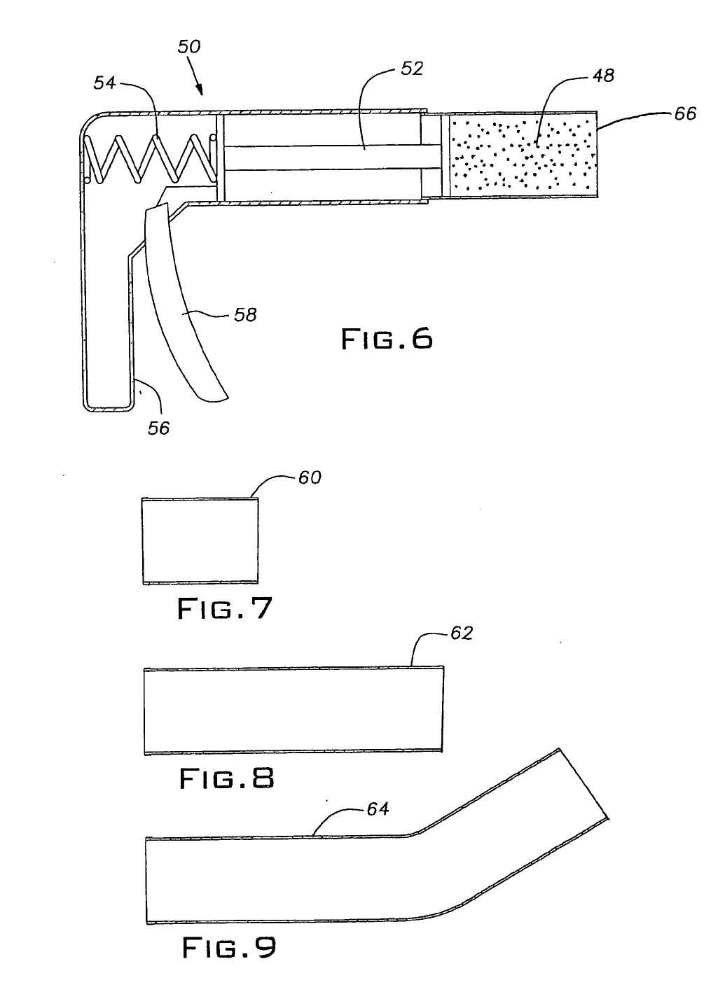

A graft material delivery tube 48 is adapted to mate with the exit port 47 of the mixing unit 30 and to receive bone graft material 72 as mentioned above and further described below. Once filled with bone graft material 72, the delivery tube 48 is detached from the exit port 47 of mixing unit 30 and is fitted to or within an injection gun 50 (see FIG. 5). The injection gun is adapted to deliver a controlled amount (at a controlled rate) of the graft material 72 to a desired graft site. As shown in FIG. 6, delivery tube 48 is preferably installed to the injection gun 50. The injection gun 50 preferably has a plunger 52, spring 54, handle 56 and trigger 58. The plunger 52 is mechanically linked to the trigger 58 such that the plunger 52 is actuated (advanced) by operation of the trigger 58. As the trigger 58 is operated, the plunger is advanced in an axial direction into the interior of delivery tube 48 to force the graft material 72 out of the nozzle end 66 of delivery tube 48. Alternatively the gun 50 can be powered by electricity, batteries, CO₂ cartridge, or other suitable mechanism.

In operation, the above-described apparatus is used as follows. Referring again to FIGS. 1 to 3, bone graft substrate material 68, such as large pieces, chips or chunks of bone, is deposited into the bone cutting chamber 18 directly above the scalloped drum 22 through the port 11. These bone chunks are preferably pressed against the drum 22 and extending scallops 23 via plunger 10 while the drum 22 is being rotated. The bone chunks are comminuted against the scallops 23 which cut out fragments 70 of bone material from the larger bone chunks. Preferably, the scallops 23 and associated apertures 25 are sized such that the resulting fragments 70 are of uniform or substantially uniform size and shape. These bone fragments 70 fall through the apertures 25 into the interior chamber or bone collection chamber 26 within the drum 22. The drum 22 is preferably rotated by an electric drill (not shown) having a suitable drill bit that is adapted to mate with a drill connector 28 at the first drum end 27 of drum 22 as described above. Less preferably, the drum 22 can be rotated via other suitable methods or means, e.g. via a hand crank attached to the connector 28. Following the comminution process, cover plate 21 is removed from the open end of the conminuting device 14, and the mixing unit 30 is mated or attached at the first mixing end 31 thereof to the open end of the comminuting device 14. The comminuted bone fragments 70 within the chamber 26 are then driven from the chamber 26 out the open drum end 29, and into the mixing chamber 32 of the mixing unit 30 via plunge plate 24. The mixing unit 30 is then detached from the comminuting unit 14, and its first mixing end 31 is sealed by attaching the first cover 51 (which preferably has the auger 34 rotatably mounted thereto) to the first mixing end 31 of the mixing unit 30.

Next, autologous tissue medium and one or more therapeutic agents are preferably injected via injector ports 36, 38, 40, 42 to provide the components of a medicinal composition according to the invention within the mixing chamber 32. Optionally, additional bone graft components can be added via the ports 36, 38, 40, 42, e.g. thrombin, cellular components, etc. In a preferred embodiment, injector ports 36, 38, 40 can be used, for example, to add autologous tissue medium as described above (such as platelet gel, tissue gel, AGF-platelet gel, bone marrow), all of which are preferably derived from the patient intraoperatively. Injector port 42 can be used, for example, to add the therapeutic agent(s). Once injected into the chamber 32, the injected materials are mixed together with the comminuted bone fragments 70 via rotation of the auger 34 to form an improved bone graft material 72 including a medicinal composition according to the invention. During this mixing step, it is important that the second cover 53 is attached over the exit port 47 of the mixing unit 30, otherwise the auger 34 could force graft material 72 out of the mixing chamber 32 prematurely through the exit port 47. Once the mixing step is complete, the second cover 53 is removed and a bone graft material delivery tube 48 is attached to the exit port flange 83. Then the improved bone graft material 72 is forced out or extruded from the mixing chamber 32, by operation of the auger 34, and into the delivery tube 48 attached to exit port 47.

The graft material delivery tube 48, now packed with the improved bone graft material 72, is then detached from the mixing unit 30 and fitted to the injection gun 50 as described above for delivery of the graft material into a patient at a graft site. The improved bone graft material is extruded from the nozzle end 66 at a desired site or defect area in the patient where bone graft material is needed, or into a spinal cage or into devices or at other locations where bone graft material is conventionally used. Delivery tubes 48 having different sizes, shapes, designs, nozzles etc., such as shown at 60,62 and 64, (FIGS. 7-9) can be used to access difficult-to-reach sites in the body. Correspondingly suitable modifications to the injection gun 50 can be made to accommodate delivery tubes 60-64 having unique or varying shapes as will be understood by a person of ordinary skill in the art.

Except as otherwise stated above, all components of the bone processing apparatus are preferably made from plastic materials, e.g. PVC, HDPE, acrylic, or other suitable plastic material. In this embodiment, the apparatus can be provided pre-sterilized in a kit as a disposable one-time use surgical device where each component of the kit has been pre-sterilized. Alternatively, where it is desired to reuse the same apparatus on multiple patients, the components can be made out of suitable metal or other materials, e.g. aluminum or surgical steel, that are capable of being sterilized via autoclaving without being damaged.

The above-described apparatus is effective to provide graft material 72 while avoiding or substantially avoiding human contact with the graft material or its constituent components or precursors while preparing the graft material. In other words, the invented apparatus allows the surgeon (and/or surgical assistants, nurses or other operating room personnel) to prepare the graft material 72 from its components without, or substantially without, touching or manipulating the components or the graft material with his hands or fingers. In this manner, the risk of cross-contamination of the graft material 72 by unwanted elements or components which may be present on the surgeon's (or other person's) hands is substantially avoided.

The bone processing apparatus described above can be used to process and provide a bone paste or bone graft material having conventional components, or having new and/or improved components or combinations of components, such as the medicinal compositions described above.

Although the hereinabove described embodiments of the invention constitute the preferred embodiments, it will be understood that modifications can be made thereto without departing from the scope of the invention as set forth in the appended claims.

What is claimed is:

1. A method of performing a surgical procedure on a patient comprising providing a medicinal composition and delivering said medicinal composition to a surgical site in said patient, said medicinal composition comprising at least 50 weight percent autologous tissue medium.

2. A method according to claim 1, said medicinal composition being in a gel state.

3. A method according to claim 2, said medicinal composition having a viscosity of at least 10 cP.

4. A method according to claim 1, said medicinal composition further comprising at least one therapeutic agent.

5. A method according to claim 1, said autologous tissue medium comprising at least one material selected from the group consisting of autologous fluids and autologous soft tissues.

6. A method according to claim 1, further comprising the steps of extracting autologous material intraoperatively from the patient's body and deriving said autologous tissue medium from said extracted autologous material.

7. A method according to claim 6, wherein said autologous material is extracted from the patient's blood, serum and/or blood-forming tissue.

8. A method according to claim 6, wherein said autologous material is extracted from the patient's fat or adipose tissue.

9. A method according to claim 1, said autologous tissue medium comprising autologous platelet gel.

10. A method according to claim 9, said autologous platelet gel having a platelet concentration in the range of 1×10⁹ to 1×10¹² platelets per mL of the autologous platelet gel.

11. A method according to claim 9, said autologous platelet gel being AGF-platelet gel.

12. A method according to claim 9, further comprising the steps of connecting the patient to a blood salvage machine during said surgical procedure, obtaining an autologous blood product from whole blood that is taken from said blood salvage machine during said surgical procedure, and preparing said autologous platelet gel from said autologous blood product.

13. A method according to claim 1, said medicinal composition being devoid of a therapeutic agent.

14. A method according to claim 4, said at least one therapeutic agent being selected from the group consisting of medications, enzymes, growth factors, chemicals and cell nutrients.

15. A method according to claim 4, said at least one therapeutic agent being a pain medication, said pain medication being present in a concentration of 1-20 weight percent in said medicinal composition.

16. A method according to claim 15, said medicinal composition further comprising a steroid, said steroid being present in a concentration of less than 15 weight percent in said medicinal composition.

17. A method according to claim 1, said medicinal composition further comprising a corticotropic preparation and/or a nerve impulse stabilizing agent.

18. A method according to claim 1, said medicinal composition further comprising living cells.

19. A method according to claim 1, wherein at least 10 weight percent of said medicinal composition is in a non-solid, flowable state.

20. A method according to claim 1, wherein said surgical procedure is a spine surgery.

21. A method according to claim 20, wherein said medicinal composition is applied within the epidural space adjacent the surgical site.

22. A method according to claim 20, said autologous tissue medium being AGF-platelet gel.

23. A method according to claim 20, said medicinal composition further comprising at least one therapeutic agent.

24. A method according to claim 23, said at least one therapeutic agent being a pain medication.

25. A method according to claim 24, said pain medication being provided in spansules for time-release administration.

26. A method according to claim 20, said medicinal composition further comprising living cells.

27. A method according to claim 1, said medicinal composition comprising 50-99 weight percent autologous platelet gel.

28. A method according to claim 1, said autologous tissue medium being derived from primary autologous material.

29. A method according to claim 1, said autologous tissue medium being derived from secondary autologous material.

30. A method of performing a surgical procedure on a patient comprising preparing a tissue graft material, implanting said tissue graft material at a graft site in the patient to provide a tissue graft, and applying a separate layer of a medicinal composition over said tissue graft, said medicinal composition comprising an autologous tissue medium.

31. A method according to claim 30, said medicinal composition being in a gel state.

32. A method according to claim 30, said medicinal composition further comprising at least one therapeutic agent.

33. A method according to claim 30, said autologous tissue medium comprising autologous material extracted intraoperatively from the patient's body.

34. A method according to claim 33, wherein said autologous material is extracted from the patient's blood, serum and/or blood-forming tissue.

35. A method according to claim 33, wherein said autologous material is extracted from the patient's fat or adipose tissue.

36. A method according to claim 30, said autologous tissue medium comprising autologous platelet gel.

37. A method according to claim 36, said autologous platelet gel being AGF-platelet gel.

38. A method according to claim 30, further comprising the step of connecting the patient to a blood salvage machine, said autologous tissue medium being prepared from whole blood that is taken from said blood salvage machine during said surgical procedure.

39. A method according to claim 30, said tissue graft being a bone graft and said tissue graft material being bone graft material, said bone graft material comprising chips, pieces and/or particles of bone and a medicinal composition, said medicinal composition comprising autologous tissue medium and at least one therapeutic agent.

40. A method according to claim 39, said at least one therapeutic agent being a pain medication.

41. A method according to claim 40, said medicinal composition further comprising a steroid.

42. A method according to claim 30, said tissue graft being a bone graft that is provided to create a spinal fusion.

43. A method according to claim 30, said tissue graft being a bone graft, said tissue graft material being bone graft material.

44. A method according to claim 30, said tissue graft being a soft tissue graft, said tissue graft material being soft tissue graft material.

45. An apparatus for preparing tissue graft material, said apparatus comprising a comminuting unit that comprises a scalloped drum having a first drum end, a second drum end, a drum wall having an interior wall surface and an exterior wall surface, and a plurality of scallops extending from the exterior wall surface of said drum wall, each said scallop having an associated aperture through said drum wall.

46. An apparatus according to claim 45, wherein said scalloped drum is made from metal.

47. An apparatus according to claim 45, said first drum end being a substantially closed drum end and said second drum end being an open drum end.

48. An apparatus according to claim 47, said comminuting unit further comprising a removable cover plate effective to seal off said open drum end during a comminuting operation.

49. An apparatus according to claim 45, said comminuting unit further comprising a casing, said scalloped drum being rotatably mounted within said casing.

50. An apparatus according to claim 49, said casing comprising at least one shield member cantilevered from an interior wall of said casing.

51. An apparatus according to claim 45, said comminuting unit further comprising a plunge plate disposed within said scalloped drum for forcing tissue fragments located within said drum out an open end thereof.

52. An apparatus according to claim 45, further comprising a mixing unit, said mixing unit comprising a mixing chamber and an auger rotatably mounted or mountable within said mixing chamber.

53. An apparatus according to claim 52, wherein said auger is made from metal.

54. An apparatus according to claim 52, said mixing unit being adapted to mate with said comminuting unit to receive tissue fragments from said scalloped drum into said mixing chamber following a comminuting operation.

55. An apparatus according to claim 52, said mixing unit further comprising a first mixing end and a cover member removably attachable to said first mixing end, said auger being rotatably mounted to said cover member, wherein said auger is introduced into the mixing chamber upon attachment of said cover member to said first mixing end of said mixing unit.

56. An apparatus according to claim 52, said mixing unit further comprising at least one injection port for injecting tissue graft material components into said mixing chamber.

57. An apparatus according to claim 52, further comprising a tissue graft material delivery tube, said mixing unit further comprising an exit port, said graft material delivery tube being adapted to mate to said exit port to receive tissue graft material therein from within said mixing chamber.

58. An apparatus according to claim 52, further comprising a tissue graft material delivery tube.

59. An apparatus according to claim 58, further comprising an injection gun adapted to mate with said tissue graft material delivery tube, and to deliver tissue graft material therein to a graft site in a patient.

60. A kit for preparing tissue graft material comprising a comminuting unit and a mixing unit, said comminuting unit comprising a scalloped drum having a first drum end, a second drum end, a drum wall having an interior wall surface and an exterior wall surface, and a plurality of scallops extending from the exterior wall surface of said drum wall, each said scallop having an associated aperture through said drum wall, said mixing unit comprising a mixing chamber and an auger mounted or mountable within said mixing chamber, said mixing unit being adapted to receive within said mixing chamber tissue fragments from said comminuting unit.

61. A kit according to claim 60, further comprising at least one tissue graft delivery tube adapted to receive tissue graft material therein.

62. A kit according to claim 60, said mixing unit adapted to mate with said comminuting unit for delivery of tissue fragments from said comminuting unit to said mixing chamber of said mixing unit.

63. A kit according to claim 61, said at least one tissue graft delivery tube being adapted to mate with said mixing unit for delivery of said tissue graft material from said mixing chamber of said mixing unit into said delivery tube.

64. A kit according to claim 60, said mixing unit further comprising at least one injector port for injecting tissue graft material components into said mixing chamber.

65. A kit according to claim 61, further comprising an injection gun, said injection gun adapted to mate with said tissue graft delivery tube and to deliver tissue graft material from within said delivery tube to a graft site in a patient.

66. A kit according to claim 60, said kit being a pre-sterilized kit.

67. A medicinal composition for application to a surgical site in a patient, said medicinal composition comprising autologous tissue medium, a pain medication and a steroid.

68. A medicinal composition according to claim 67, said autologous tissue medium being autologous platelet gel.

69. A medicinal composition according to claim 67, said medicinal composition being effective to inhibit formation of scar tissue postoperatively at the surgical site.

70. A medicinal composition according to claim 67, said medicinal composition being effective to promote hemostasis postoperatively at the surgical site.

71. A medicinal composition according to claim 67, said autologous tissue medium being derived from primary autologous material.

72. A medicinal composition according to claim 67, said autologous tissue medium being derived from secondary autologous material.
